(19) United States
(12) Patent Application Publication
(10) Pub. No.: US 2014/0121239 A1
(43) Pub. Date: May 1, 2014

(54) METHOD OF TREATING LUNG ADENOCARCINOMA

(71) Applicant: EXELIXIS, INC., South San Francisco, CA (US)

(72) Inventor: DANA T. AFTAB, San Rafael, CA (US)

(73) Assignee: EXELIXIS, INC., South San Francisco, CA (US)

(21) Appl. No.: 14/021,614

(22) Filed: Sep. 9, 2013

Related U.S. Application Data
(60) Provisional application No. 61/698,143, filed on Sep. 7, 2012.

(51) Int. Cl. C07D 215/22

(52) U.S. Cl. CPC C07D 215/22
USPC 514/312

(57) ABSTRACT

This invention is directed to the treatment of cancer in a patient, particularly a patient with lung adenocarcinoma, and more particularly a patient with KIF5B-RET fusion-positive non-small cell lung cancer, with an inhibitor of MET, VEGF, and RET which is a compound of Formula I:

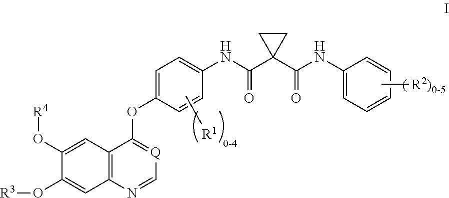

or a pharmaceutically acceptable salt thereof.

### CROSS-REFERENCE TO RELATED APPLICATIONS

This application claims the benefit of priority of U.S. Provisional Application No. 61/698,143, filed Sep. 7, 2012, the entire contents of which is incorporated herein by reference.

### SEQUENCE LISTING

This application incorporates by reference in its entirety the Sequence Listing entitled “SequenceListing.txt” (EX12-001C-US_ST25.txt, 1.86 KB) which was created on Dec. 27, 2013 and filed herewith on Dec. 31, 2013.

### FIELD OF THE INVENTION

This invention is directed to the treatment of cancer, particularly lung adenocarcinoma, using an inhibitor of MET, VEGFR, and RET.

### BACKGROUND OF THE INVENTION

Lung cancer is the leading cause of cancer-related mortality worldwide. Recent developments in targeted therapies have led to a treatment paradigm shift in non-small-cell lung cancer (NSCLC). Mok T S, Wu Y L, Thongprasert S, Yang C H, Chu D T, Saijo N, Sunpaweravong P, Han B, Margono B, Ichinose Y, Nishiwaki Y, Ohe Y, Yang J J, Chewaskulyong B, Jiang H, Duffield E L, Watkins C L, Armour A A, Fukuoka M. Gefitinib or carboplatin-paclitaxel in pulmonary adenocarcinoma. N Engl J. Med. 2009 Sep. 3; 361(10):947-57. Maemondo M, Inoue A, Kobayashi K, Sugawara S, Oizumi S, Isobe H, Gemma A, Harada M, Yoshizawa H, Kinoshita I, Fujita Y, Okinaga S, Hirano H, Yoshimori K, Harada T, Ogura T, Ando M, Miyazawa H, Tanaka T, Saijo Y, Hagiwara K, Morita S, Nukiwa T; North-East Japan Study Group. Gefitinib or chemotherapy for non-small-cell lung cancer with mutated EGFR. N Engl J. Med. 2010 Jun. 24; 362(25):2380-8. Epidermal growth factor receptor (EGFR) tyrosine kinase inhibitors (TKIs), gefitinib and erlotinib, and the anaplastic lymphoma kinase (ALK) T M, crizotinib, have shown clinical activity in NSCLC patients with EGFR mutations or ALK gene rearrangements. Kwak E L, Bang Y J, Camidge D R, Shaw A T, Solomon B, Maki R G, Ou S H, Dezube B J, Janne P A, Costa D B, Varella-Garcia M, Kim W H, Lynch T J, Fidias P, Stubbs H, Engelman J A, Sequist L V, Tan W, Gandhi L, Mino-Kenudson M, Wei G C, Shreeve S M, Ratain M J, Settleman J, Christensen J G, Haber D A, Wilner K, Salgia R, Shapiro G I, Clark J W, Iafrate A J. Anaplastic lymphoma kinase inhibition in non-small-cell lung cancer. N Engl J. Med. 2010 Oct. 28; 363(18):1693-703. In addition, ROS1 gene rearrangement has been reported in approximately 2% of patients with NSCLC, and clinical activity has been reported using crizotinib in this patient subgroup. Bergethon K, Shaw A T, Ou S H, Katayama R, Lovly C M, McDonald N T, Massion P P, Siwak-Tapp C, Gonzalez A, Fang R, Mark E J, Batten J M, Chen H, Wilner K D, Kwak E L, Clark J W, Carbone D P, Ji H, Engelman J A, Mino-Kenudson M, Pao W, Iafrate A J. ROS 1 rearrangements define a unique molecular class of lung cancers. J Clin Oncol. 2012 Mar. 10; 30(8):863-70. Shaw A T, Camidge, Engelman J A, Solomon B J, Kwak E L, Clark J W, Salgia R, Shapiro, Bang Y J, Tan W, Tye L, Wilner K D, Stephenson P, Varella-Garcia M, Bergethon K, Iafrate A J, Ou S H. Clinical activity of crizotinib in advanced non-small cell lung cancer (NSCLC) harboring ROS1 gene rearrangement. J Clin Oncol. 2012 30 (suppl; abstr 7508). Fusion of the KIF5B (the kinesin family 5B) gene and the RET oncogene has been recently reported as a driver mutation in 1-2% of NSCLC patients and are a focus as a therapeutic target. Kohno T, Ichikawa H, Totoki Y, Yasuda K, Hiramoto M, Nammo T, Sakamoto H, Tsuta K, Furuta K, Shimada Y, Iwakawa R, Ogiwara H, Oike T, Enari M, Schetter A J, Okayama H, Haugen A, Skaug V, Chiku S, Yamanaka I, Arai Y, Watanabe S, Sekine I, Ogawa S, Harris C C, Tsuda H, Yoshida T, Yokota J, Shibata T. KIF5B-RET fusions in lung adenocarcinoma. Nat. Med. 2012 Feb. 12; 18(3):375-7. Takeuchi K, Soda M, Togashi Y, Suzuki R, Sakata S, Hatano S, Asaka R, Hamanaka W, Ninomiya H, Uehara H, Lim Choi Y, Satoh Y, Okumura S, Nakagawa K, Mano H, Ishikawa Y. RET, ROS 1 and ALK fusions in lung cancer. Nat. Med. 2012 Feb. 12; 18(3):378-81. Lipson D, Capelletti M, Yelensky R, Otto G, Parker A, Jarosz M, Curran J A, Balasubramanian S, Bloom T, Brennan K W, Donahue A, Downing S R, Frampton G M, Garcia L, Juhn F, Mitchell K C, White E, White J, Zwirko Z, Peretz T, Nechushtan H, Soussan-Gutman L, Kim J, Sasaki H, Kim H R, Park S I, Ercan D, Sheehan C E, Ross J S, Cronin M T, Janne P A, Stephens P J. Identification of new ALK and RET gene fusions from colorectal and lung cancer biopsies. Nat. Med. 2012 Feb. 12; 18(3):382-4. Thus, it is becoming more important to identify key driver genes in NSCLC and to develop therapies for each genomic subset of patients.

### SUMMARY OF THE INVENTION

These and other needs are met by the present invention which is directed to a method for treating lung adenocarcinoma using an inhibitor of MET, VEGFR, and RET. The method comprises administering a therapeutically effective amount of a compound that modulates MET, VEGFR, and RET to a patient in need of such treatment. In one embodiment, the lung adenocarcinoma is non-small cell lung cancer (NSCLC). More particularly, the lung adenocarcinoma is KIF5B-RET fusion-positive NSCLC.

In one aspect, the present invention is directed to a method for treating NSCLC in a patient in need of such treatment, comprising administering a therapeutically effective amount of a compound that simultaneously modulates MET, VEGFR, and RET to the patient.

In one embodiment of this and other aspects, the dual acting MET/VEGFR/RET inhibitor is a compound of Formula I

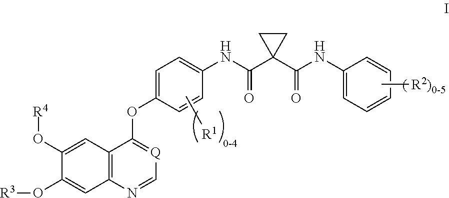

or a pharmaceutically acceptable salt thereof, wherein:
R¹ is halo;
R² is halo;
R³ is (C₁-C₆)alkyl;
R⁴ is (C₁-C₆)alkyl; and
Q is CH or N.

In another embodiment, the compound of Formula I is a compound of Formula Ia

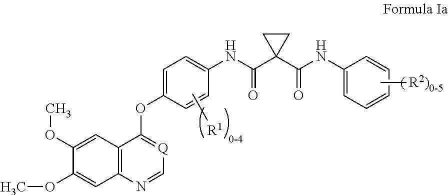

or a pharmaceutically acceptable salt thereof, wherein:
R¹ is halo;
R² is halo; and
Q is CH or N.

In another embodiment, the compound of Formula I is compound 1:

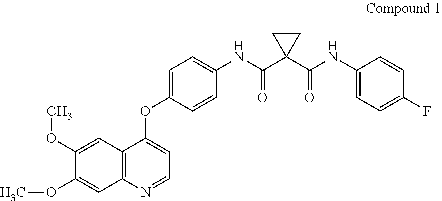 [SMILES: COc1cc2nccc(Oc3ccc(NC(=O)C4(C(=O)Nc5ccc(F)cc5)CC4)cc3)c2cc1OC]

or a pharmaceutically acceptable salt thereof. Compound 1 is known as N-(4-{[6,7-bis(methyloxy)quinolin-4-yl]oxy}phenyl)-N′-(4-fluorophenyl)cyclopropane-1,1-dicarboxamide and by the name Cabozantinib.

Compound 1 is a potent inhibitor of c-MET, RET, and VEGFR2. Yakes F M, Chen J, Tan J, Yamaguchi K, Shi Y, Yu P, Qian F, Chu F, Bentzien F, Camilla B, Orf J, You A, Laird A D, Engst S, Lee L, Lesch J, Chou Y C, Joly A H. Cabozantinib (XL184), a novel MET and VEGFR2 inhibitor, simultaneously suppresses metastasis, angiogenesis, and tumor growth. Mol Cancer Ther. 2011 December; 10(12):2298-308. In preclinical studies, Compound 1-mediated inhibition of kinase activity produced rapid and robust regression of tumor vasculature, tumor invasiveness and metastasis, and prolonged survival. Sennino B. Inhibition of tumor invasiveness by c-MET/VEGFR blockade. Presented at: Gordon Research Conference: Angiogenesis; Aug. 2-7, 2009; Newport, R I. You W K, Falcon B, Hashizume H et al. Exaggerated regression of blood vessels, hypoxia, and apoptosis in tumors after c-MET and VEGFR inhibition. Am J Pathol, submitted.

In another embodiment, the compound of Formula I, Ia, or Compound 1 is administered as a pharmaceutical composition comprising a pharmaceutically acceptable additive, diluent, or excipient.

In another aspect, the invention provides a method for treating KIF5B-RET fusion-positive NSCLC, comprising administering a therapeutically effective amount of a pharmaceutical composition comprising a Compound of Formula I or the malate salt of a Compound of Formula I or another pharmaceutically acceptable salt of a Compound of Formula I, to a patient in need of such treatment. In a specific embodiment, the Compound of Formula I is Compound 1 or the malate salt of Compound 1.

In another aspect, the invention provides a method for treating a lung adenocarcinoma which is KIF5B-RET fusion positive non-small cell lung cancer in a patient in need of such treatment, comprising administering to the patient an effective amount of compound 1:

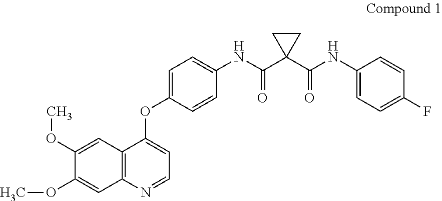 [SMILES: COc1cc2nccc(Oc3ccc(NC(=O)C4(C(=O)Nc5ccc(F)cc5)CC4)cc3)c2cc1OC]

or a pharmaceutically acceptable salt thereof.

### BRIEF DESCRIPTION OF THE FIGURES

FIG. 1A depicts inhibition of phosphorylation of RET in vivo in TT-tumor bearing animals that were administered a single escalating doses of Compound 1 or water vehicle.

FIG. 1B depicts the effect of the administration of a single oral dose of Compound 1 (100 mg/kg) on mice bearing TT tumors on phosphorylation levels and total RET, AKT, and ERK in tumor lysates.

FIG. 1C provides densitometric quantitation of the duration of inhibition of phosphorylation of RET versus plasma concentrations of Compound 1, along with representative Western blot images.

FIG. 2A shows that Compound 1 inhibits TT xenograft tumor growth that correlating with serum reductions in calcitonin in nu/nu mice bearing TT tumors that were orally administered once daily water vehicle (□) or cabozantinib at 3 mg/kg (∇), 10 mg/kg (◯), 30 mg/kg (♦), or 60 mg/kg (⋄) for 21 days.

FIG. 2B shows circulating calcitonin levels determined in serum preparations from whole blood collected after the final indicated doses.

FIG. 2C shows significant and dose dependent decreases in levels of phosphorylated RET and phosphorylated MET in the absence of reduced levels of total protein after treatment with compound 1.

FIG. 3 depicts the response of a patient with KIF5B-RET fusion-positive NCSLC to Compound 1. Computed tomography scans of the chest were obtained at baseline (FIG. 3A) and after 9 weeks (FIG. 3B) of Compound 1.

FIG. 4A depicts KIF5B-RET genome PCR and Sanger sequencing from pre- and post-treatment tumor samples.

FIG. 4B depicts KIF5B-RET RT-PCR and Sanger sequencing from post-treatment tumor sample.

FIG. 4C depicts break-apart FISH at the RET locus in tumor cells.

### DETAILED DESCRIPTION OF THE INVENTION

### Abbreviations and Definitions

The following abbreviations and terms have the indicated meanings throughout this application.

| Abbreviation | Meaning |
|---|---|
| Ac | Acetyl |
| Br | Broad |
| ° C. | Degrees Celsius |
| c- | Cyclo |
| CBZ | CarboBenZoxy = benzyloxycarbonyl |
| d | Doublet |
| dd | Doublet of doublet |
| dt | Doublet of triplet |
| DCM | Dichloromethane |
| DME | 1,2-dimethoxyethane |
| DMF | N,N-Dimethylformamide |
| DMSO | dimethyl sulfoxide |
| EI | Electron Impact ionization |
| G | Gram(s) |
| h or hr | Hour(s) |
| HPLC | High pressure liquid chromatography |
| L | Liter(s) |
| M | Molar or molarity |
| m | Multiplet |
| Mg | Milligram(s) |
| MHz | Megahertz (frequency) |
| Min | Minute(s) |
| mL | Milliliter(s) |
| μL | Microliter(s) |
| μM | Micromole(s) or micromolar |
| mM | Millimolar |
| Mmol | Millimole(s) |
| Mol | Mole(s) |
| MS | Mass spectral analysis |
| N | Normal or normality |
| nM | Nanomolar |
| NMR | Nuclear magnetic resonance spectroscopy |
| q | Quartet |
| RT | Room temperature |
| s | Singlet |
| t or tr | Triplet |
| TFA | Trifluoroacetic acid |
| THF | Tetrahydrofuran |
| TLC | Thin layer chromatography |

The symbol “-” means a single bond, “=” means a double bond.

When chemical structures are depicted or described, unless explicitly stated otherwise, all carbons are assumed to have hydrogen substitution to conform to a valence of four. For example, in the structure on the left-hand side of the schematic below there are nine hydrogens implied. The nine hydrogens are depicted in the right-hand structure. Sometimes a particular atom in a structure is described in textual formula as having a hydrogen or hydrogens as substitution (expressly defined hydrogen), for example, —CH₂CH₂—. It is understood by one of ordinary skill in the art that the aforementioned descriptive techniques are common in the chemical arts to provide brevity and simplicity to description of otherwise complex structures.

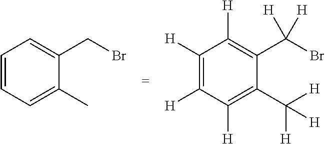

If a group “R” is depicted as “floating” on a ring system, as for example in the formula:

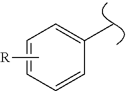

then, unless otherwise defined, a substituent “R” may reside on any atom of the ring system, assuming replacement of a depicted, implied, or expressly defined hydrogen from one of the ring atoms, so long as a stable structure is formed.

If a group “R” is depicted as floating on a fused ring system, as for example in the formulae:

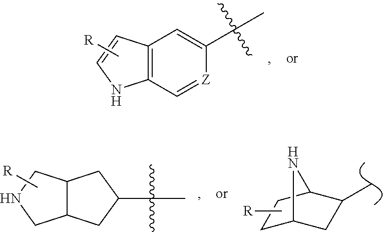

then, unless otherwise defined, a substituent “R” may reside on any atom of the fused ring system, assuming replacement of a depicted hydrogen (for example the —NH— in the formula above), implied hydrogen (for example as in the formula above, where the hydrogens are not shown but understood to be present), or expressly defined hydrogen (for example where in the formula above, “Z” equals ═CH—) from one of the ring atoms, so long as a stable structure is formed. In the example depicted, the “R” group may reside on either the 5-membered or the 6-membered ring of the fused ring system. When a group “R” is depicted as existing on a ring system containing saturated carbons, as for example in the formula:

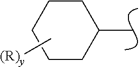

where, in this example, “y” can be more than one, assuming each replaces a currently depicted, implied, or expressly defined hydrogen on the ring; then, unless otherwise defined, where the resulting structure is stable, two “R's” may reside on the same carbon. A simple example is when R is a methyl group; there can exist a geminal dimethyl on a carbon of the depicted ring (an “annular” carbon). In another example, two R's on the same carbon, including that carbon, may form a ring, thus creating a spirocyclic ring (a “spirocyclyl” group) structure with the depicted ring as for example in the formula:

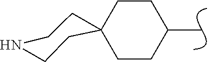

“Halogen” or “halo” refers to fluorine, chlorine, bromine or iodine.

“Yield” for each of the reactions described herein is expressed as a percentage of the theoretical yield.

“Patient” for the purposes of the present invention includes humans and other animals, particularly mammals, and other organisms. Thus the methods are applicable to both human therapy and veterinary applications. In another embodiment the patient is a mammal, and in another embodiment the patient is human.

A “pharmaceutically acceptable salt” of a compound means a salt that is pharmaceutically acceptable and that possesses the desired pharmacological activity of the parent compound. It is understood that the pharmaceutically acceptable salts are non-toxic. Additional information on suitable pharmaceutically acceptable salts can be found in Remington's Pharmaceutical Sciences, 17^{th} ed., Mack Publishing Company, Easton, Pa., 1985, which is incorporated herein by reference or S. M. Berge, et al., “Pharmaceutical Salts,” J. Pharm. Sci., 1977; 66:1-19 both of which are incorporated herein by reference.

Examples of pharmaceutically acceptable acid addition salts include those formed with inorganic acids such as hydrochloric acid, hydrobromic acid, sulfuric acid, nitric acid, phosphoric acid, and the like; as well as organic acids such as acetic acid, trifluoroacetic acid, propionic acid, hexanoic acid, cyclopentanepropionic acid, glycolic acid, pyruvic acid, lactic acid, oxalic acid, maleic acid, malonic acid, succinic acid, fumaric acid, tartaric acid, malic acid, citric acid, benzoic acid, cinnamic acid, 3-(4-hydroxybenzoyl)benzoic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, p-toluenesulfonic acid, and salicylic acid and the like.

“Prodrug” refers to compounds that are transformed (typically rapidly) in vivo to yield the parent compound of the above formulae, for example, by hydrolysis in blood. Common examples include, but are not limited to, ester and amide forms of a compound having an active form bearing a carboxylic acid moiety. Examples of pharmaceutically acceptable esters of the compounds of this invention include, but are not limited to, alkyl esters (for example with between about one and about six carbons) the alkyl group is a straight or branched chain. Acceptable esters also include cycloalkyl esters and arylalkyl esters such as, but not limited to benzyl. Examples of pharmaceutically acceptable amides of the compounds of this invention include, but are not limited to, primary amides, and secondary and tertiary alkyl amides (for example with between about one and about six carbons). Amides and esters of the compounds of the present invention may be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

“Therapeutically effective amount” is an amount of a compound of the invention, that when administered to a patient, ameliorates a symptom of the disease. A therapeutically effective amount is intended to include an amount of a compound alone or in combination with other active ingredients effective to modulate c-Met, and/or VEGFR², or effective to treat or prevent cancer. The amount of a compound of the invention which constitutes a “therapeutically effective amount” will vary depending on the compound, the disease state and its severity, the age of the patient to be treated, and the like. The therapeutically effective amount can be determined by one of ordinary skill in the art having regard to their knowledge and to this disclosure.

“Treating” or “treatment” of a disease, disorder, or syndrome, as used herein, includes (i) preventing the disease, disorder, or syndrome from occurring in a human, i.e. causing the clinical symptoms of the disease, disorder, or syndrome not to develop in an animal that may be exposed to or predisposed to the disease, disorder, or syndrome but does not yet experience or display symptoms of the disease, disorder, or syndrome; (ii) reversing or inhibiting the disease, disorder, or syndrome, i.e., arresting its development; and (iii) relieving the disease, disorder, or syndrome, i.e., causing regression of the disease, disorder, or syndrome. As is known in the art, adjustments for systemic versus localized delivery, age, body weight, general health, sex, diet, time of administration, drug interaction and the severity of the condition may be necessary, and will be ascertainable with routine experience.

### EMBODIMENTS

In one embodiment the compound of Formula I is the compound of Formula Ia:

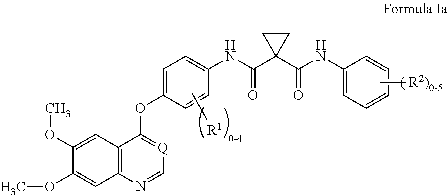

or a pharmaceutically acceptable salt thereof, wherein:
R¹ is halo;
R² is halo; and
Q is CH or N.

In another embodiment, the compound of Formula I is Compound 1:

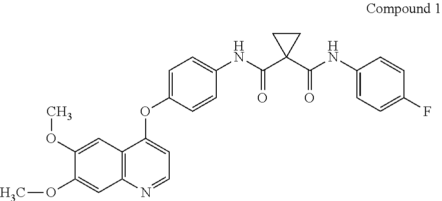 [SMILES: COc1cc2nccc(Oc3ccc(NC(=O)C4(C(=O)Nc5ccc(F)cc5)CC4)cc3)c2cc1OC]

or a pharmaceutically acceptable salt thereof. As indicated previously, compound 1 is referred to herein as N-(4-{[6,7-bis(methyloxy)quinolin-4-yl]oxy}phenyl)-N′-(4-fluorophenyl)cyclopropane-1,1-dicarboxamide. WO 2005/030140 which is incorporated herein by reference in its entirety discloses Compound 1 and describes how it is made (Example 12, 37, 38, and 48) and also discloses the therapeutic activity of this compound to inhibit, regulate and/or modulate the signal transduction of kinases, (Assays, Table 4, entry 289). Example 48 is on paragraph [0353] in WO 2005/030140.

In other embodiments, the compound of Formula I, Ia, or Compound 1, or a pharmaceutically acceptable salt thereof, is administered as a pharmaceutical composition, wherein the pharmaceutical composition additionally comprises a pharmaceutically acceptable carrier, excipient, or diluent. In a specific embodiment, the Compound of Formula I is Compound 1.

The compound of Formula I, Formula Ia and Compound I, as described herein, includes both the recited compounds as well as individual isomers and mixtures of isomers. In each instance, the compound of Formula I includes the pharmaceutically acceptable salts, hydrates, and/or solvates of the recited compounds and any individual isomers or mixture of isomers thereof.

In other embodiments, the compound of Formula I, Ia, or Compound 1 can be the (L)-malate salt. The malate salt of the Compound of Formula I and of Compound 1 is disclosed in PCT/US2010/021194 and U.S. Ser. No. 61/325,095, both of which are incorporated herein by reference.

In other embodiments, the compound of Formula I is the (D)-malate salt.

In other embodiments, the compound of Formula Ia is the malate salt.

In other embodiments, the compound of Formula Ia is the (L)-malate salt.

In other embodiments, Compound 1 is the (D)-malate salt.

In other embodiments, Compound 1 is the malate salt.

In other embodiments, Compound 1 is the (L)-malate salt.

In another embodiment, the malate salt is in the crystalline N-1 form or the N-2 form of the (L) malate salt and/or the (D) malate salt of Compound 1 as disclosed in U.S. patent Application Ser. No. 61/325,095. Also see WO 2008/083319, incorporated by reference in its entirety, for the properties of crystalline enantiomers, including the N-1 and/or the N-2 crystalline forms of the malate salt of Compound 1. Methods of making and characterizing such forms are fully described in PCT/US10/021,194, which is incorporated herein by reference in its entirety.

In another embodiment, the invention is directed to a method for reversing or inhibiting NSCLC, comprising administering to a patient in need of such treatment a therapeutically effective amount of a compound of Formula I in any of the embodiments disclosed herein. In a specific embodiment, the Compound of Formula I is Compound 1.

In another embodiment, the invention is directed to a method for reversing or inhibiting KIF5B-RET fusion-positive NSCLC, comprising administering to a patient in need of such treatment a therapeutically effective amount of a compound of Formula I in any of the embodiments disclosed herein. In a specific embodiment, the Compound of Formula I is Compound 1.

In another embodiment, the compound of Formula I is administered before, concurrently, or subsequent to one or more other treatments. In another embodiment, the compound of Formula I is administered subsequent to one or more treatments. “Treatment” means any of the treatment options are available to the skilled artisan, including surgery, chemotherapeutic agents, hormone therapies, antibodies, immunotherapies, radioactive iodine therapy, and radiation. In particular, “treatment’ means another chemotherapeutic agent or antibody.

Thus, in another embodiment, the compound of Formula I is administered post-cisplatin and/or gemcitabine treatment.

In another embodiment, the compound of Formula I is administered post-doectaxel treatment.

In another embodiment, the compound of Formula I is administered post HER-2 antibody treatment. In another embodiment, the HER-2 antibody is trastuzumab.

In another embodiment, the compound of Formula I is administered post-cisplatin and/or gemacitabine and/or docetaxel treatment.

In another embodiment, the Compound of Formula I, Ia, or Compound 1 or a pharmaceutically acceptable salt thereof is administered orally once daily as a tablet or capsule. In these and other embodiments, the Compound of Formula I is Compound 1.

In another embodiment, Compound 1 is administered orally as its free base or malate salt as a capsule or tablet.

in another embodiment, Compound 1 is administered orally once daily as its free base or as the malate salt as a capsule or tablet containing up to 100 mg of Compound 1.

In another embodiment, Compound 1 is administered orally once daily as its free base or as the malate salt as a capsule or tablet containing IOU mg of Compound 1.

In another embodiment, Compound 1 is administered orally once daily as its free base or as the malate salt as a capsule or tablet containing 95 mg of Compound 1.

In another embodiment, Compound 1 is administered orally once daily as its free base or as the malate salt as a capsule or tablet containing 90 mg of Compound 1.

In another embodiment, Compound 1 is administered orally once daily as its free base or as the malate salt as a capsule or tablet containing 85 mg of Compound 1.

In another embodiment, Compound 1 is administered orally once daily as its free base or as the malate salt as a capsule or tablet containing 80 mg of Compound 1.

In another embodiment, Compound 1 is administered orally once daily as its free base or as the malate salt as a capsule or tablet containing 75 mg of Compound 1.

In another embodiment, Compound 1 is administered orally once daily as its free base or as the malate salt as a capsule or tablet containing 70 mg of Compound 1.

In another embodiment, Compound 1 is administered orally once daily as its free base or as the malate salt as a capsule or tablet containing 65 mg of Compound 1.

In another embodiment, Compound 1 is administered orally once daily as its free base or as the malate salt as a capsule or tablet containing 60 mg of Compound 1.

In another embodiment, Compound 1 is administered orally once daily as its free base or as the malate salt as a capsule or tablet containing 55 mg of Compound 1.

In another embodiment, Compound 1 is administered orally once daily as its free base or as the malate salt as a capsule or tablet containing 50 mg of Compound 1.

In another embodiment, Compound 1 is administered orally once daily as its free base or as the malate salt as a capsule or tablet containing 45 mg of Compound 1.

In another embodiment, Compound 1 is administered orally once daily as its free base or as the malate salt as a capsule or tablet containing 40 mg of Compound 1.

In another embodiment, Compound 1 is administered orally once daily as its free base or as the malate salt as a capsule or tablet containing 30 mg of Compound 1.

In another embodiment, Compound 1 is administered orally once daily as its free base or as the malate salt as a capsule or tablet containing 25 mg of Compound 1.

In another embodiment, Compound 1 is administered orally once daily as its free base or as the malate salt as a capsule or tablet containing 20 mg of Compound 1.

In another embodiment, Compound 1 is administered orally once daily as its free base or as the malate salt as a capsule or tablet containing 15 mg of Compound 1.

In another embodiment, Compound 1 is administered orally once daily as its free base or as the malate salt as a capsule or tablet containing 10 mg of Compound 1.

In another embodiment, Compound 1 is administered orally once daily as its free base or as the malate salt as a capsule or tablet containing 5 mg of Compound 1.

In another embodiment, Compound 1 is administered as its free base or malate salt orally once daily as a tablet as provided in the following table.

| Ingredient | (% w/w) |
|---|---|
| Compound 1 | 31.68 |
| Microcrystalline Cellulose | 38.85 |
| Lactose anhydrous | 19.42 |
| Hydroxypropyl Cellulose | 3.00 |
| Croscarmellose Sodium | 3.00 |
| Total Intra-granular | 95.95 |
| Silicon dioxide, Colloidal | 0.30 |
| Croscarmellose Sodium | 3.00 |
| Magnesium Stearate | 0.75 |
| Total | 100.00 |

In another embodiment, Compound 1 is administered orally as its free base or malate salt once daily as a tablet as provided in the following table.

| Ingredient | (% w/w) |
|---|---|
| Compound 1 | 25.0-33.3 |
| Microcrystalline Cellulose | q.s |
| Hydroxypropyl Cellulose | 3 |
| Poloxamer | 0-3 |
| Croscarmellose Sodium | 6.0 |
| Colloidal Silicon Dioxide | 0.5 |
| Magnesium Stearate | 0.5-1.0 |
| Total | 100 |

In another embodiment, Compound 1 is administered orally as its free base or malate salt once daily as a tablet as provided in the following table.

| | Theoretical Quantity |
|---|---|
| Ingredient | (mg/unit dose) |
| Compound 1 | 100.0 |
| Microcrystalline Cellulose PH-102 | 155.4 |
| Lactose Anhydrous 60M | 77.7 |
| Hydroxypropyl Cellulose, EXF | 12.0 |
| Croscarmellose Sodium | 24 |
| Colloidal Silicon Dioxide | 1.2 |
| Magnesium Stearate (Non-Bovine) | 3.0 |
| Opadry Yellow | 16.0 |
| Total | 416 |

Any of the tablet formulations provided above can be adjusted according to the dose of Compound 1 desired. Thus, the amount of each of the formulation ingredients can be proportionally adjusted to provide a table formulation containing various amounts of Compound 1 as provided in the previous paragraphs. In another embodiment, the formulations can contain 20, 40, 60, or 80 mg of Compound 1.

In these and other embodiments, the invention provides a method for inhibiting or reversing the progress of abnormal cell growth in a mammal, comprising administering Compound 1 or a pharmaceutically acceptable salt thereof, wherein the abnormal cell growth is cancer mediated by KIF5B-RET. In one embodiment, the cancer is lung adenocarcinoma. In another, the lung adenocarcinoma is non-small cell lung cancer. In another, the lung adenocarcinoma is KIF5B-RET fusion-positive non-small cell lung cancer. In another embodiment, Compound 1 or a pharmaceutically acceptable salt thereof is administered as a pharmaceutical composition comprising Compound 1 or a pharmaceutically acceptable salt thereof and at least one pharmaceutically acceptable carrier. In another embodiment, the compound of Formula I is administered subsequent to another form of treatment. In another embodiment, Compound 1 is administered post-cisplatin and/or gemcitabine treatment. In another embodiment, Compound 1 is administered post-doectaxel treatment. In another embodiment, Compound 1 is administered post-cisplatin and/or gemcitabine and/or docetaxel treatment.

### Administration

Administration of the compound of Formula I, Formula Ia, or Compound 1, or a pharmaceutically acceptable salt thereof, in pure form or in an appropriate pharmaceutical composition, can be carried out via any of the accepted modes of administration or agents for serving similar utilities. Thus, administration can be, for example, orally, nasally, parenterally (intravenous, intramuscular, or subcutaneous), topically, transdermally, intravaginally, intravesically, intracistemally, or rectally, in the form of solid, semi-solid, lyophilized powder, or liquid dosage forms, such as for example, tablets, suppositories, pills, soft elastic and hard gelatin dosages (which can be in capsules or tablets), powders, solutions, suspensions, or aerosols, or the like, specifically in unit dosage forms suitable for simple administration of precise dosages.

The compositions will include a conventional pharmaceutical carrier or excipient and a compound of Formula I as the/an active agent, and, in addition, may include carriers and adjuvants, etc.

Adjuvants include preserving, wetting, suspending, sweetening, flavoring, perfuming, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

If desired, a pharmaceutical composition of the compound of Formula I may also contain minor amounts of auxiliary substances such as wetting or emulsifying agents, pH buffering agents, antioxidants, and the like, such as, for example, citric acid, sorbitan monolaurate, triethanolamine oleate, butylalted hydroxytoluene, etc.

The choice of composition depends on various factors such as the mode of drug administration (e.g., for oral administration, compositions in the form of tablets, pills or capsules) and the bioavailability of the drug substance. Recently, pharmaceutical compositions have been developed especially for drugs that show poor bioavailability based upon the principle that bioavailability can be increased by increasing the surface area i.e., decreasing particle size. For example, U.S. Pat. No. 4,107,288 describes a pharmaceutical composition having particles in the size range from 10 to 1,000 nm in which the active material is supported on a crosslinked matrix of macromolecules. U.S. Pat. No. 5,145,684 describes the production of a pharmaceutical composition in which the drug substance is pulverized to nanoparticles (average particle size of 400 nm) in the presence of a surface modifier and then dispersed in a liquid medium to give a pharmaceutical composition that exhibits remarkably high bioavailability.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

One specific route of administration is oral, using a convenient daily dosage regimen that can be adjusted according to the degree of severity of the disease-state to be treated.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid dosage forms as described above can be prepared with coatings and shells, such as enteric coatings and others well known in the art. They may contain pacifying agents, and can also be of such composition that they release the active compound or compounds in a certain part of the intestinal tract in a delayed manner. Examples of embedded compositions that can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., the compound of Formula I, or a pharmaceutically acceptable salt thereof, and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administration are, for example, suppositories that can be prepared by mixing the compound of Formula I with, for example, suitable non-irritating excipients or carriers such as cocoa butter, polyethyleneglycol or a suppository wax, which are solid at ordinary temperatures but liquid at body temperature and therefore, melt while in a suitable body cavity and release the active component therein.

Dosage forms for topical administration of the compound of Formula I include ointments, powders, sprays, and inhalants. The active component is admixed under sterile conditions with a physiologically acceptable carrier and any preservatives, buffers, or propellants as may be required. Ophthalmic compositions, eye ointments, powders, and solutions are also contemplated as being within the scope of this disclosure.

Compressed gases may be used to disperse the compound of Formula I in aerosol form. Inert gases suitable for this purpose are nitrogen, carbon dioxide, etc.

Generally, depending on the intended mode of administration, the pharmaceutically acceptable compositions will contain about 1% to about 99% by weight of a compound(s) of Formula I, or a pharmaceutically acceptable salt thereof, and 99% to 1% by weight of a suitable pharmaceutical excipient. In one example, the composition will be between about 5% and about 75% by weight of a compound(s) of Formula I, Formula Ia, or Compound 1, or a pharmaceutically acceptable salt thereof, with the rest being suitable pharmaceutical excipients.

Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of Formula I, or a pharmaceutically acceptable salt thereof, for treatment of a disease-state in accordance with the teachings of this disclosure.

The compounds of this disclosure, or their pharmaceutically acceptable salts or solvates, are administered in a therapeutically effective amount which will vary depending upon a variety of factors including the activity of the specific compound employed, the metabolic stability and length of action of the compound, the age, body weight, general health, sex, diet, mode and time of administration, rate of excretion, drug combination, the severity of the particular disease-states, and the host undergoing therapy. The compound of Formula I, Formula Ia, or Compound 1, can be administered to a patient at dosage levels in the range of about 0.1 to about 1,000 mg per day. For a normal human adult having a body weight of about 70 kilograms, a dosage in the range of about 0.01 to about 100 mg per kilogram of body weight per day is an example. The specific dosage used, however, can vary. For example, the dosage can depend on a number of factors including the requirements of the patient, the severity of the condition being treated, and the pharmacological activity of the compound being used. The determination of optimum dosages for a particular patient is well known to one of ordinary skill in the art.

In other embodiments, the compound of Formula I, Formula Ia, or Compound 1, can be administered to the patient concurrently with other cancer treatments. Such treatments include other cancer chemotherapeutics, hormone replacement therapy, radiation therapy, or immunotherapy, among others. The choice of other therapy will depend on a number of factors including the metabolic stability and length of action of the compound, the age, body weight, general health, sex, diet, mode and time of administration, rate of excretion, drug combination, the severity of the particular disease-states, and the host undergoing therapy.

### Preparation of Compound 1

### Preparation Of N-(4-{[6,7-bis(methyloxy)quinolin-4-yl]oxy}phenyl)-N′-(4-fluorophenyl)cyclopropane-1,1-dicarboxamide and the (L)-malate salt thereof

The synthetic route used for the preparation of N-(4-[6,7-bis(methyloxy)quinolin-4-yl]oxy}phenyl)-N′-(4-fluorophenyl)cyclopropane-1,1-dicarboxamide and the (L)-malate salt thereof is depicted in Scheme 1:

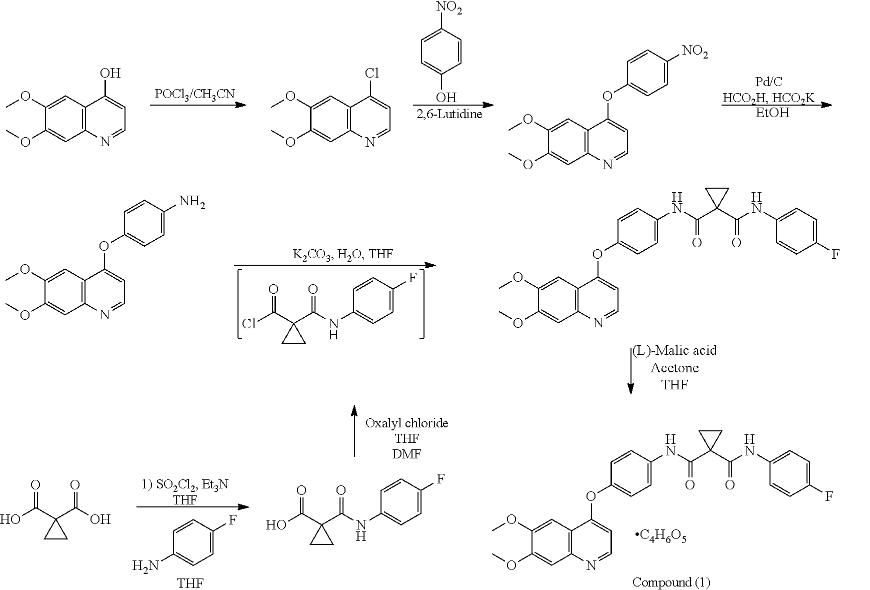

### Preparation of 4-Chloro-6,7-dimethoxy-quinoline

A reactor was charged sequentially with 6,7-dimethoxy-quinoline-4-ol (10.0 kg) and acetonitrile (64.0 L). The resulting mixture was heated to approximately 65° C. and phosphorus oxychloride (POCl₃, 50.0 kg) was added. After the addition of POCl₃, the temperature of the reaction mixture was raised to approximately 80° C. The reaction was deemed complete (approximately 9.0 hours) when less than 2 percent of the starting material remained (in process high-performance liquid chromotography [HPLC] analysis). The reaction mixture was cooled to approximately 10° C. and then quenched into a chilled solution of dichloromethane (DCM, 238.0 kg), 30% NH₄OH (135.0 kg), and ice (440.0 kg). The resulting mixture was warmed to approximately 14° C., and phases were separated. The organic phase was washed with water (40.0 kg) and concentrated by vacuum distillation to remove the solvent (approximately 190.0 kg). Methyl-t-butyl ether (MTBE, 50.0 kg) was added to the batch, and the mixture was cooled to approximately 10° C., during which time the product crystallized out. The solids were recovered by centrifugation, washed with n heptane (20.0 kg), and dried at approximately 40° C. to afford the title compound (8.0 kg).

### Preparation of 6,7-Dimethyl-4-(4-nitro-phenoxy)-quinoline

A reactor was sequentially charged with 4-chloro-6,7-dimethoxy-quinoline (8.0 kg), 4 nitrophenol (7.0 kg), 4 dimethylaminopyridine (0.9 kg), and 2,6 lutidine (40.0 kg). The reactor contents were heated to approximately 147° C. When the reaction was complete (less than 5 percent starting material remaining as determined by in process HPLC analysis, approximately 20 hours), the reactor contents were allowed to cool to approximately 25° C. Methanol (26.0 kg) was added, followed by potassium carbonate (3.0 kg) dissolved in water (50.0 kg). The reactor contents were stirred for approximately 2 hours. The resulting solid precipitate was filtered, washed with water (67.0 kg), and dried at 25° C. for approximately 12 hours to afford the title compound (4.0 kg).

### Preparation of 4-(6,7-Dimethoxy-quinoline-4-yloxy)-phenylamine

A solution containing potassium formate (5.0 kg), formic acid (3.0 kg), and water (16.0 kg) was added to a mixture of 6,7-dimethoxy-4-(4-nitro-phenoxy)-quinoline (4.0 kg), 10 percent palladium on carbon (50 percent water wet, 0.4 kg) in tetrahydrofuran (THF, 40.0 kg) that had been heated to approximately 60° C. The addition was carried out such that the temperature of the reaction mixture remained approximately 60° C. When the reaction was deemed complete as determined using in-process HPLC analysis (less than 2 percent starting material remaining, typically 1 5 hours), the reactor contents were filtered. The filtrate was concentrated by vacuum distillation at approximately 35° C. to half of its original volume, which resulted in the precipitation of the product. The product was recovered by filtration, washed with water (12.0 kg), and dried under vacuum at approximately 50° C. to afford the title compound (3.0 kg; 97 percent area under curve (AUC)).

### Preparation of 1-(4-Fluoro-phenylcarbamoyl)-cyclopropanecarboxylic acid

Triethylamine (8.0 kg) was added to a cooled (approximately 4° C.) solution of commercially available cyclopropane-1,1-dicarboxylic acid (2 1, 10.0 kg) in THF (63.0 kg) at a rate such that the batch temperature did not exceed 10° C. The solution was stirred for approximately 30 minutes, and then thionyl chloride (9.0 kg) was added, keeping the batch temperature below 10° C. When the addition was complete, a solution of 4-fluoroaniline (9.0 kg) in THF (25.0 kg) was added at a rate such that the batch temperature did not exceed 10° C. The mixture was stirred for approximately 4 hours and then diluted with isopropyl acetate (87.0 kg). This solution was washed sequentially with aqueous sodium hydroxide (2.0 kg dissolved in 50.0 L of water), water (40.0 L), and aqueous sodium chloride (10.0 kg dissolved in 40.0 L of water). The organic solution was concentrated by vacuum distillation followed by the addition of heptane, which resulted in the precipitation of solid. The solid was recovered by centrifugation and then dried at approximately 35° C. under vacuum to afford the title compound. (10.0 kg).

### Preparation of 1-(4-Fluoro-phenylcarbamoyl)-cyclopropanecarbonyl chloride

Oxalyl chloride (1.0 kg) was added to a solution of 1-(4-fluoro-phenylcarbamoyl)-cyclopropanecarboxylic acid (2.0 kg) in a mixture of THF (11 kg) and N,N-dimethylformamide (DMF; 0.02 kg) at a rate such that the batch temperature did not exceed 30° C. This solution was used in the next step without further processing.

### Preparation of N-(4-{[6,7-bis(methyloxy)quinolin-4-yl]oxy}phenyl)-N′-(4-fluorophenyl)cyclopropane-1,1-dicarboxamide

The solution from the previous step containing 1-(4-fluoro-phenylcarbamoyl)-cyclopropanecarbonyl chloride was added to a mixture of 4-(6,7-dimethoxy-quinoline-4-yloxy)-phenylamine (3.0 kg) and potassium carbonate (4.0 kg) in THF (27.0 kg) and water (13.0 kg) at a rate such that the batch temperature did not exceed 30° C. When the reaction was complete (in typically 10 minutes), water (74.0 kg) was added. The mixture was stirred at 15-30° C. for approximately 10 hours, which resulted in the precipitation of the product. The product was recovered by filtration, washed with a pre-made solution of THF (11.0 kg) and water (24.0 kg), and dried at approximately 65° C. under vacuum for approximately 12 hours to afford the title compound (free base, 5.0 kg). ¹H NMR (400 MHz, d₆-DMSO): δ 10.2 (s, 1H), 10.05 (s, 1H), 8.4 (s, 1H), 7.8 (m, 2H), 7.65 (m, 2H), 7.5 (s, 1H), 7.35 (s, 1H), 7.25 (m, 2H), 7.15 (m, 2H), 6.4 (s, 1H), 4.0 (d, 6H), 1.5 (s, 4H). LC/MS: M+H=502.

### Preparation of N-(4-{[6,7-bis(methyloxy)quinolin-4-yl]oxy}phenyl)-N′-(4-fluorophenyl)cyclopropane-1,1-dicarboxamide, (L) malate salt

A solution of L-malic acid (2.0 kg) in water (2.0 kg) was added to a solution of Cyclopropane-1,1-dicarboxylic acid [4-(6,7-dimethoxy-quinoline-4-yloxy)-phenyl]-amide(4-fluoro-phenyl)-amide free base (1 5, 5.0 kg) in ethanol, maintaining a batch temperature of approximately 25° C. Carbon (0.5 kg) and thiol silica (0.1 kg) were then added, and the resulting mixture was heated to approximately 78° C., at which point water (6.0 kg) was added. The reaction mixture was then filtered, followed by the addition of isopropanol (38.0 kg), and was allowed to cool to approximately 25° C. The product was recovered by filtration and washed with isopropanol (20.0 kg), and dried at approximately 65° C. to afford the title compound (5.0 kg).

### Alternative Preparation of N-(4-{[6,7-Bis(methyloxy)quinolin-4-yl]oxy}phenyl)-N′-(4-fluorophenyl)cyclopropane-1,1-dicarboxamide and the (L)-malate salt thereof

An alternative synthetic route that can be used for the preparation of N-(4-{[6,7-bis(methyloxy)quinolin-4-yl]oxy}phenyl)-N′-(4-fluorophenyl)cyclopropane-1,1-dicarboxamide and the (L)-malate salt thereof is depicted in Scheme 2, as described in PCT/US2012/024591, the entire contents of which is incorporated herein by reference.

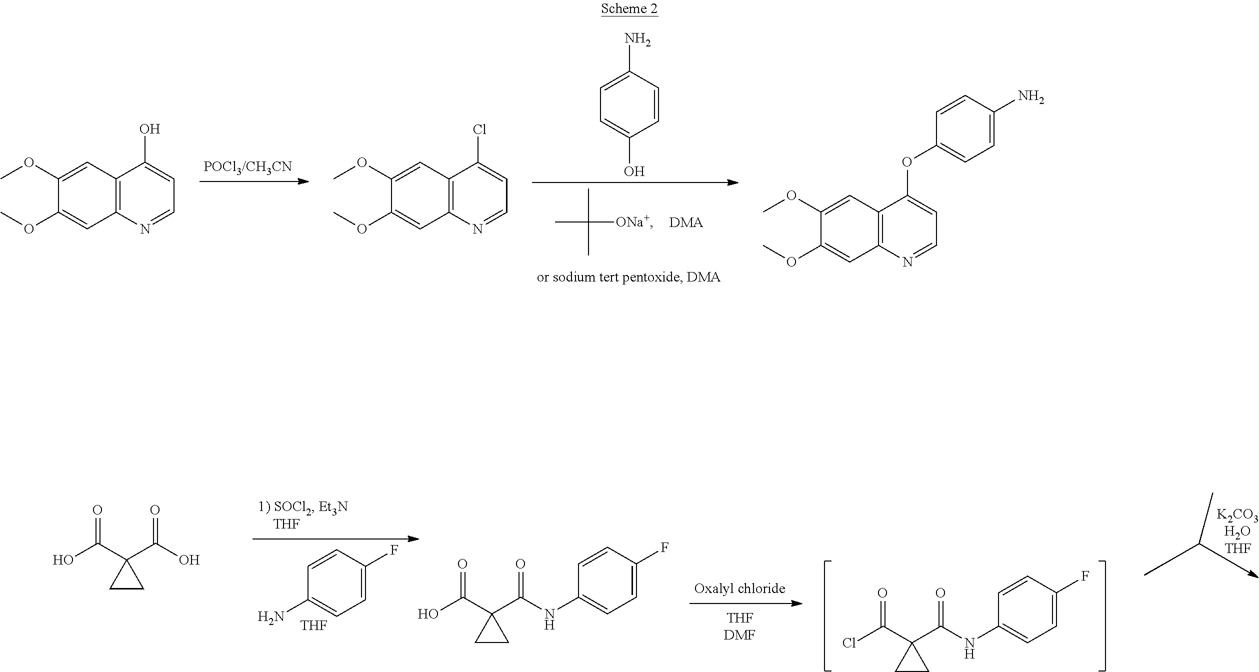

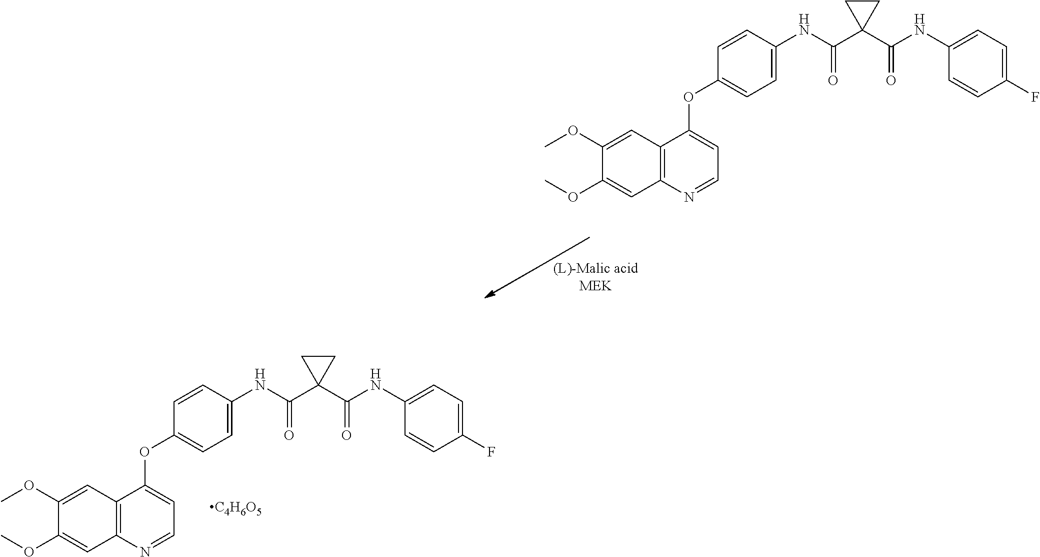

### Preparation of 4-Chloro-6,7-dimethoxy-quinoline

A reactor was charged sequentially with 6,7-dimethoxy-quinoline-4-ol (47.0 kg) and acetonitrile (318.8 kg). The resulting mixture was heated to approximately 60° C. and phosphorus oxychloride (POCl₃, 130.6 kg) was added. After the addition of POCl₃, the temperature of the reaction mixture was raised to approximately 77° C. The reaction was deemed complete (approximately 13 hours) when less than 3% of the starting material remained (in-process high-performance liquid chromatography [HPLC] analysis). The reaction mixture was cooled to approximately 2-7° C. and then quenched into a chilled solution of dichloromethane (DCM, 482.8 kg), 26 percent NH₄OH (251.3 kg), and water (900 L). The resulting mixture was warmed to approximately 20-25° C., and phases were separated. The organic phase was filtered through a bed of AW hyflo super-cel NF (Celite; 5.4 kg) and the filter bed was washed with DCM (118.9 kg). The combined organic phase was washed with brine (282.9 kg) and mixed with water (120 L). The phases were separated and the organic phase was concentrated by vacuum distillation with the removal of solvent (approximately 95 L residual volume). DCM (686.5 kg) was charged to the reactor containing organic phase and concentrated by vacuum distillation with the removal of solvent (approximately 90 L residual volume). Methyl t-butyl ether (MTBE, 226.0 kg) was then charged and the temperature of the mixture was adjusted to −20 to −25° C. and held for 2.5 hours resulting in solid precipitate which was then filtered and washed with n-heptane (92.0 kg), and dried on a filter at approximately 25° C. under nitrogen to afford the title compound. (35.6 kg).

### Preparation of 4-(6,7-Dimethoxy-quinoline-4-yloxy)-phenylamine

4-Aminophenol (24.4 kg) dissolved in N,N-dimethylacetamide (DMA, 184.3 kg) was charged to a reactor containing 4-chloro-6,7-dimethoxyquinoline (35.3 kg), sodium t-butoxide (21.4 kg) and DMA (167.2 kg) at 20-25° C. This mixture was then heated to 100-105° C. for approximately 13 hours. After the reaction was deemed complete as determined using in-process HPLC analysis (less than 2 percent starting material remaining), the reactor contents were cooled at 15-20° C. and water (pre-cooled, 2-7° C., 587 L) charged at a rate to maintain 15-30° C. temperature. The resulting solid precipitate was filtered, washed with a mixture of water (47 L) and DMA (89.1 kg) and finally with water (214 L). The filter cake was then dried at approximately 25° C. on filter to yield crude 4-(6,7-dimethoxy-quinoline-4-yloxy)-phenylamine (59.4 kg wet, 41.6 kg dry calculated based on LOD). Crude 4-(6,7-dimethoxy-quinoline-4-yloxy)-phenylamine was refluxed (approximately 75° C.) in a mixture of tetrahydrofuran (THF, 211.4 kg) and DMA (108.8 kg) for approximately 1 hour and then cooled to 0-5° C. and aged for approximately 1 hour after which time the solid was filtered, washed with THF (147.6 kg) and dried on a filter under vacuum at approximately 25° C. to yield 4-(6,7-dimethoxy-quinoline-4-yloxy)-phenylamine (34.0 kg).

### Alternative Preparation of 4-(6,7-Dimethoxy-quinoline-4-yloxy)-phenylamine

4-chloro-6,7-dimethoxyquinoline (34.8 kg) and 4-aminophenol (30.8 kg) and sodium tert pentoxide (1.8 equivalents) 88.7 kg, 35 weight percent in THF) were charged to a reactor, followed by N,N-dimethylacetamide (DMA, 293.3 kg). This mixture was then heated to 105-115° C. for approximately 9 hours. After the reaction was deemed complete as determined using in-process HPLC analysis (less than 2 percent starting material remaining), the reactor contents were cooled at 15-25° C. and water (315 kg) was added over a two hour period while maintaining the temperature between 20-30° C. The reaction mixture was then agitated for an additional hour at 20-25° C. The crude product was collected by filtration and washed with a mixture of 88 kg water and 82.1 kg DMA, followed by 175 kg water. The product was dried on a filter drier for 53 hours. The LOD showed less than 1 percent w/w.

In an alternative procedure, 1.6 equivalents of sodium tert-pentoxide were used and the reaction temperature was increased from 110-120° C. In addition, the cool down temperature was increased to 35-40° C. and the starting temperature of the water addition was adjusted to 35-40° C., with an allowed exotherm to 45° C.

### Preparation of 1-(4-Fluoro-phenylcarbamoyl)-cyclopropanecarboxylic acid

Triethylamine (19.5 kg) was added to a cooled (approximately 5° C.) solution of cyclopropane-1,1-dicarboxylic acid (24.7 kg) in THF (89.6 kg) at a rate such that the batch temperature did not exceed 5° C. The solution was stirred for approximately 1.3 hours, and then thionyl chloride (23.1 kg) was added, keeping the batch temperature below 10° C. When the addition was complete, the solution was stirred for approximately 4 hours keeping temperature below 10° C. A solution of 4-fluoroaniline (18.0 kg) in THF (33.1 kg) was then added at a rate such that the batch temperature did not exceed 10° C. The mixture was stirred for approximately 10 hours after which the reaction was deemed complete. The reaction mixture was then diluted with isopropyl acetate (218.1 kg). This solution was washed sequentially with aqueous sodium hydroxide (10.4 kg, 50 percent dissolved in 119 L of water) further diluted with water (415 L), then with water (100 L) and finally with aqueous sodium chloride (20.0 kg dissolved in 100 L of water). The organic solution was concentrated by vacuum distillation (100 L residual volume) below 40° C. followed by the addition of n-heptane (171.4 kg), which resulted in the precipitation of solid. The solid was recovered by filtration and washed with n-heptane (102.4 kg), resulting in wet, crude 1-(4-fluoro-phenylcarbamoyl)-cyclopropanecarboxylic acid (29.0 kg). The crude, 1-(4-fluoro-phenylcarbamoyl)-cyclopropanecarboxylic acid was dissolved in methanol (139.7 kg) at approximately 25° C. followed by the addition of water (320 L) resulting in slurry which was recovered by filtration, washed sequentially with water (20 L) and n-heptane (103.1 kg) and then dried on the filter at approximately 25 C under nitrogen to afford the title compound (25.4 kg).

### Preparation of 1-(4-Fluoro-phenylcarbamoyl)-cyclopropanecarbonyl chloride

Oxalyl chloride (12.6 kg) was added to a solution of 1-(4-fluoro-phenylcarbamoyl)-cyclopropanecarboxylic acid (22.8 kg) in a mixture of THF (96.1 kg) and N,N-dimethylformamide (DMF; 0.23 kg) at a rate such that the batch temperature did not exceed 25° C. This solution was used in the next step without further processing.

### Alternative Preparation of 1-(4-Fluoro-phenylcarbamoyl)-cyclopropanecarbonyl chloride

A reactor was charged with 1-(4-fluoro-phenylcarbamoyl)-cyclopropanecarboxylic acid (35 kg), 344 g DMF, and 175 kg THF. The reaction mixture was adjusted to 12-17° C. and then to the reaction mixture was charged 19.9 kg of oxalyl chloride over a period of 1 hour. The reaction mixture was left stirring at 12-17° C. for 3 to 8 hours. This solution was used in the next step without further processing.

### Preparation of cyclopropane-1,1-dicarboxylic acid [4-(6,7-dimethoxy-quinoline-4-yloxy)-phenyl]-amide(4-fluoro-phenyl)-amide

The solution from the previous step containing 1-(4-fluoro-phenylcarbamoyl)-cyclopropanecarbonyl chloride was added to a mixture of compound 4-(6,7-dimethoxy-quinoline-4-yloxy)-phenylamine (23.5 kg) and potassium carbonate (31.9 kg) in THF (245.7 kg) and water (116 L) at a rate such that the batch temperature did not exceed 30° C. When the reaction was complete (in approximately 20 minutes), water (653 L) was added. The mixture was stirred at 20-25° C. for approximately 10 hours, which resulted in the precipitation of the product. The product was recovered by filtration, washed with a pre-made solution of THF (68.6 kg) and water (256 L), and dried first on a filter under nitrogen at approximately 25° C. and then at approximately 45° C. under vacuum to afford the title compound (41.0 kg, 38.1 kg, calculated based on LOD).

### Alternative Preparation of cyclopropane-1,1-dicarboxylic acid [4-(6,7-dimethoxy-quinoline-4-yloxy)-phenyl]-amide(4-fluoro-phenyl)-amide

A reactor was charged with 4-(6,7-dimethoxy-quinoline-4-yloxy)-phenylamine (35.7 kg, 1 equivalent), followed by 412.9 kg THF. To the reaction mixture was charged a solution of 48.3 K₂CO₃ in 169 kg water. The acid chloride solution of described in the Alternative Preparation of 1-(4-Fluoro-phenylcarbamoyl)-cyclopropanecarbonyl chloride above was transferred to the reactor containing 4-(6,7-dimethoxy-quinoline-4-yloxy)-phenylamine while maintaining the temperature between 20-30° C. over a minimum of two hours. The reaction mixture was stirred at 20-25° C. for a minimum of three hours. The reaction temperature was then adjusted to 30-25° C. and the mixture was agitated. The agitation was stopped and the phases of the mixture were allowed to separate. The lower aqueous phase was removed and discarded. To the remaining upper organic phase was added 804 kg water. The reaction was left stirring at 15-25° C. for a minimum of 16 hours.

The product precipitated. The product was filtered and washed with a mixture of 179 kg water and 157.9 kg THF in two portions. The crude product was dried under a vacuum for at least two hours. The dried product was then taken up in 285.1 kg THF. The resulting suspension was transferred to reaction vessel and agitated until the suspension became a clear (dissolved) solution, which required heating to 30-35° C. for approximately 30 minutes. 456 kg water was then added to the solution, as well as 20 kg SDAG-1 ethanol (ethanol denatured with methanol over two hours. The mixture was agitated at 15-25° C. fir at least 16 hours. The product was filtered and washed with a mixture of 143 kg water and 126.7 THF in two portions. The product was dried at a maximum temperature set point of 40° C.

In an alternative procedure, the reaction temperature during acid chloride formation was adjusted to 10-15° C. The recrystallization temperature was changed from 15-25° C. to 45-50° C. for 1 hour and then cooled to 15-25° C. over 2 hours.

### Preparation of cyclopropane-1,1-dicarboxylic acid [4-(6,7-dimethoxy-quinoline-4-yloxy)-phenyl]-amide(4-fluoro-phenyl)-amide, malate salt

Cyclopropane-1,1-dicarboxylic acid [4-(6,7-dimethoxy-quinoline-4-yloxy)-phenyl]-amide(4-fluoro-phenyl)-amide(1-5; 13.3 kg), L-malic acid (4.96 kg), methyl ethyl ketone (MEK: 188.6 kg) and water (37.3 kg) were charged to a reactor and the mixture was heated to reflux (approximately 74° C.) for approximately 2 hours. The reactor temperature was reduced to 50 to 55° C. and the reactor contents were filtered. These sequential steps described above were repeated two more times starting with similar amounts of starting material (13.3 kg), L-Malic acid (4.96 kg), MEK (198.6 kg) and water (37.2 kg). The combined filtrate was azeotropically dried at atmospheric pressure using MEK (1133.2 kg) (approximate residual volume 711 L; KF≦0.5% w/w) at approximately 74° C. The temperature of the reactor contents was reduced to 20 to 25° C. and held for approximately 4 hours resulting in solid precipitate which was filtered, washed with MEK (448 kg) and dried under vacuum at 50° C. to afford the title compound (45.5 kg).

### Alternative Preparation of cyclopropane-1,1-dicarboxylic acid [4-(6,7-dimethoxy-quinoline-4-yloxy)-phenyl]-amide(4-fluoro-phenyl)-amide, (L) malate salt

Cyclopropane-1,1-dicarboxylic acid [4-(6,7-dimethoxy-quinoline-4-yloxy)-phenyl]-amide(4-fluoro-phenyl)-amide(47.9 kg), L-malic acid (17.2), 658.2 kg methyl ethyl ketone, and 129.1 kg water (37.3 kg) were charged to a reactor and the mixture was heated 50-55° C. for approximately 1-3 hours, and then at 55-60° C. for an additional 4-5 hours. The mixture was clarified by filtration through a 1 μm cartridge. The reactor temperature was adjusted to 20-25° C. and vacuum distilled with a vacuum at 150-200 mm Hg with a maximum jacket temperature of 55° C. to the volume range of 558-731 L.

The vacuum distillation was performed two more times with the charge of 380 kg and 380.2 kg methyl ethyl ketone, respectively. After the third distillation, the volume of the batch was adjusted to 18 v/w of cyclopropane-1,1-dicarboxylic acid [4-(6,7-dimethoxy-quinoline-4-yloxy)-phenyl]-amide(4-fluoro-phenyl)-amide by charging 159.9 kg methyl ethyl ketone to give a total volume of 880 L. An additional vacuum distillation was carried out by adjusting 245.7 methyl ethyl ketone. The reaction mixture was left with moderate agitation at 20-25° C. for at least 24 hours. The product was filtered and washed with 415.1 kg methyl ethyl ketone in three portions. The product was dried under a vacuum with the jacket temperature set point at 45° C.

In an alternative procedure, the order of addition was changed so that a solution of 17.7 kg L-malic acid dissolved in 129.9 kg water was added to cyclopropane-1,1-dicarboxylic acid [4-(6,7-dimethoxy-quinoline-4-yloxy)-phenyl]amide(4-fluoro-phenyl)-amide(48.7 kg) in methyl ethyl ketone (673.3 kg).

### Biological Example

### Compound 1 is a Potent Inhibitor of RET In Vitro

Compound 1 has been previously shown to be an ATP-competitive inhibitor of MET (IC₅₀=1.3 nmol/L) and VEGFR2 (IC₅₀=0.035 nmol/L) when profiled against a protein kinase panel of 270 human kinases. See Yakes, Mol Cancer Ther. 2011 December; 10(12):2298-308. Compound 1 is also a potent inhibitor of RET with a biochemical IC₅₀ value of 5.2 nmol/L. RET-activating kinase domain mutations M918T and Y791F—known to be associated with hereditary and sporadic medullary thyroid carcinoma—were also inhibited by Compound 1 with IC₅₀ values of 27 and 1173 nmol/L, respectively. Moreover, Compound 1 was not active against the RET mutant V804L (IC₅₀>5000 nmol/L), which is known to render resistance to RET inhibitors. In cellular assays, Compound 1 inhibited RET autophosphorylation in TT cells, a calcitonin-expressing human medullary thyroid carcinoma cell line that harbors an activating C634W mutant of RET, with an IC₅₀ value of 85 nmol/L. The effect of Compound 1 on the growth of TT cells that were grown in 10% serum for 72 hours (3 days) was also investigated. Compound 1 treatment resulted in dose-dependent inhibition of proliferation with an IC₅₀ value of 94 nmol/L.

### Biological Example

### Compound 1 Inhibits Ligand-Independent Phosphorylation of RET In Vivo

TT-tumor bearing animals were administered single escalating doses of Compound 1 or water vehicle, and tumors were collected 4 h post dose. Levels of phosphorylated and total RET and were determined in pooled lysates by Western immunoblot analysis. In a separate study, mice bearing TT tumors were administered a single oral dose of cabozantinib (100 mg/kg) or water vehicle, and levels of phosphorylated and total RET, AKT, and ERK in tumor lysates were determined at the indicated time points post dose. Densitometric quantitation of the duration of inhibition of phosphorylation of RET versus plasma concentrations of cabozantinib. Representative Western blot images are shown.

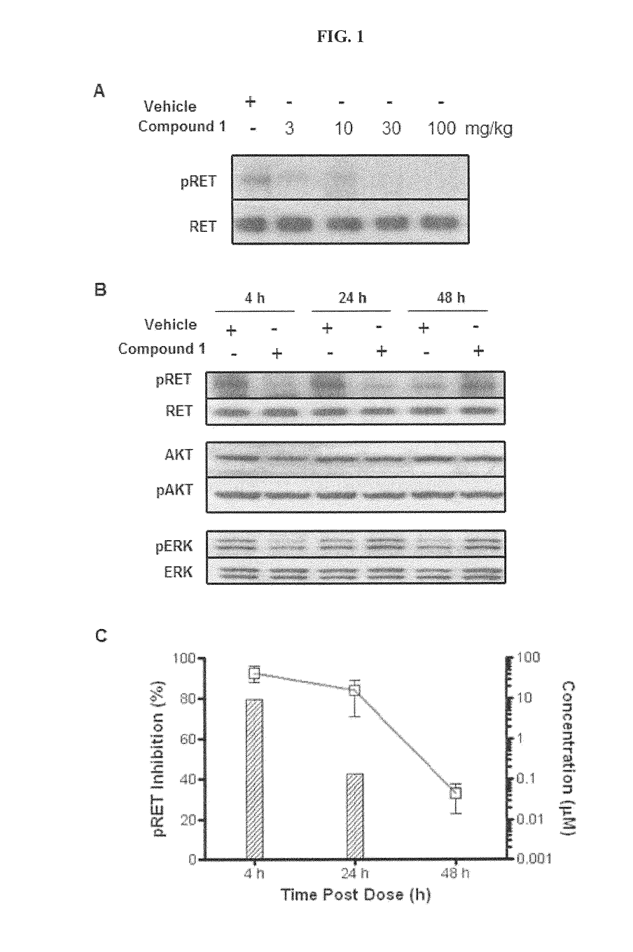

Single ascending oral dose administration of Compound 1 resulted in dose-dependent inhibition of phosphorylation of RET in the absence of reduced RET protein levels in TT xenograft tumors as depicted in FIG. 1A. This result is consistent with data demonstrating the sensitivity of multiple medullary thyroid carcinoma cell lines to pharmacologic inhibitors selective for RET and RET knockdown by siRNA. Based on the dose-response relationship the predicted plasma concentration that results in 50% inhibition (IC₅₀) of phosphorylation of RET in this xenograft model is approximately 7 μmol/L. In a subsequent study, a single 100-mg/kg oral dose resulted in inhibition of phosphorylation of RET 4 to 24 hours post dose in TT xenograft tumors, as depicted in FIG. 1B. This effect was reversible as RET phosphorylation returned to basal levels by 48 hours after treatment as depicted in FIG. 1C. In addition, Compound 1 reduced phosphorylation levels of AKT and ERK 4 to 24 hours post dose, which is consistent with inhibition of RET-mediated activation of the RAS/RAF/MAPK pathway. Plasma concentrations of Compound 1 associated with maximal and sustained inhibition of RET (15 μmol/L), AKT and ERK (42 μmol/L), respectively.

### Biological Example

### Compound 1 Inhibits TT Tumor Growth

The ability of Compound 1 to inhibit the growth of TT xenograft tumors was evaluated in nu/nu mice over a period of time corresponding to exponential tumor growth. Nu/nu mice bearing TT tumors were orally administered once daily water vehicle (□) or cabozantinib at 3 mg/kg (∇), 10 mg/kg (◯), 30 mg/kg (♦), or 60 mg/kg (⋄) for 21 days. Tumor weights were determined twice weekly. Data points represent the mean tumor weight (in milligrams) and SE for each treatment group. Circulating calcitonin levels were determined in serum preparations from whole blood collected after the final indicated doses (* indicates a significant, P<0.05, reduction in circulating calcitonin when compared to serum samples from vehicle-treated control animals).

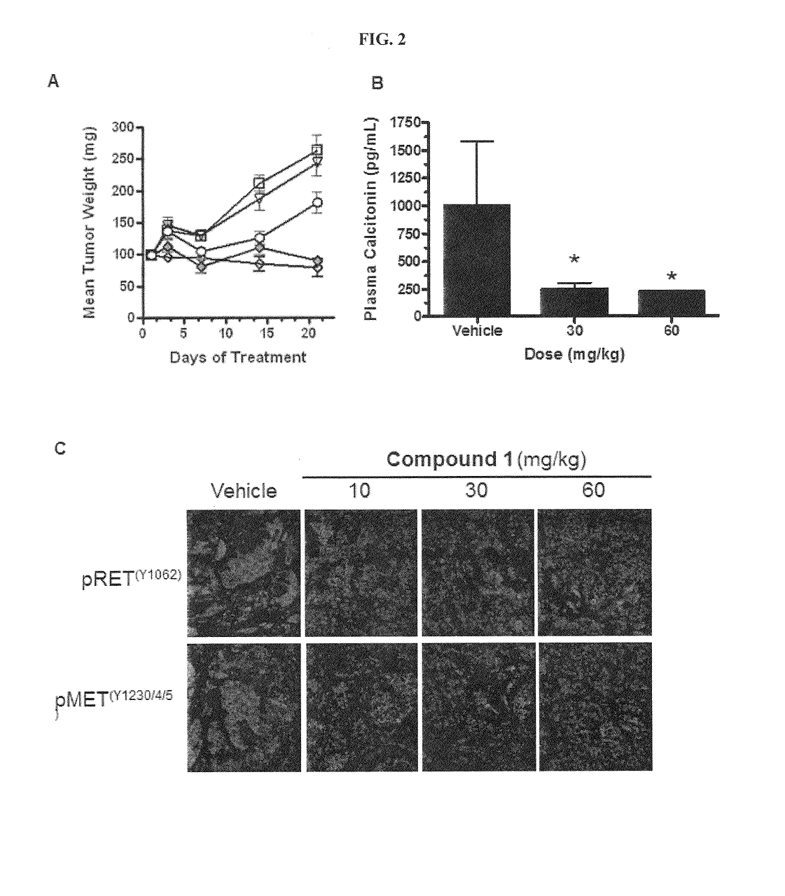

Compound 1 inhibits TT xenograft tumor growth that correlates with serum reductions in calcitonin, as depicted in FIG. 2A with dose-dependent inhibition achieved for the 10- and 30-mg/kg doses. Furthermore, stable disease was observed at the 30- and 60-mg/kg doses that was associated with peak cyclical plasma concentrations of 3,000 to 45,000 nmol/L. Subchronic administration of Compound 1 was well tolerated as determined by stable body weights collected throughout the dosing period. Given that TT xenograft tumors are known to secrete high amounts of human calcitonin that correlates with tumor size, serum concentrations of circulating calcitonin were determined at the end of the dosing period. Serum from vehicle-treated control animals exhibited high levels of circulating calcitonin that was markedly reduced (75%; P<0.005) at both the 30- and 60-mg/kg doses when compared to vehicle control animals, as depicted in FIG. 2B. Moreover, this reduction in circulating plasma calcitonin correlated with TT tumor growth inhibition described above. Immunohistochemical analysis of tumors revealed significant and dose dependent decreases in levels of phosphorylated RET and MET as depicted in FIG. 2C in the absence of reduced levels of total protein. Furthermore, Compound 1 treatment also resulted in dose dependent reductions in Ki67 and CD31 in viable tumor tissue indicating a negative impact on markers of cellular proliferation and vascularity as summarized in Table 1.

TABLE 1
Summary of Histochemical Analyses of TT Xenograft Tumors

| | RET^{(Y1062)} | | MET^{(Y1230/4/5)} | | CD31 | | Ki67 | |
|---|---|---|---|---|---|---|---|---|
| Cabozantinib Dose | Relative | Inhibition | Relative | Inhibition | Positive | Reduction | Positive | Reduction |
| (mg/kg) | Area | (%)^{a} | Area | (%)^{a} | Cells (%) | (%)^{a} | Cells (%) | (%)^{a} |
| Vehicle | 32.7 ± 2.6 | na | 27.4 ± 2.6 | na | 55.3 ± 6.9 | na | 26.6 ± 3.9 | na |
| 3 | 25.2 2.9 | 23 | 21.6 ± 2.7 | 21 | 35.9 ± 4.7 | 35 | 20.7 ± 2.6 | 22 |
| 10 | 17.4 1.9 | 47 | 17.2 ± 2.3 | 37 | 33.5 ± 4.9 | 39 | 19.4 ± 3.0 | 27 |
| 30 | 12.5 2.0 | 62 | 10.7 ± 1.5 | 61 | 26.4 ± 6.4 | 52 | 14.3 ± 3.9 | 46 |
| 60 | 9.7 2.1 | 70 | 8.2 ± 2.2 | 70 | 22.7 ± 8.6 | 59 | 8.1 ± 2.5 | 69 |

^{a}P < 0.05 unless otherwise indicated

### Case Study

A 51-year-old Japanese woman who was a former smoker presented in April 2009 for evaluation of a right pleural effusion. Computed tomography (CT) scans of the chest revealed a mass in the right middle lobe and right pleural effusion. Cytological examination of the pleural effusion revealed adenocarcinoma and EGFR was determined to be wild-type using high resolution melting analysis. A systemic workup showed no evidence of distant metastasis. There was also no tumor in either the neck or thyroid on the CT scans. The patient was diagnosed as having stage IIIB (cT4N0M0, 6th edition of the International System for Staging Lung cancer) adenocarcinoma of the lung. She was treated with 4 cycles of cisplatin and gemcitabine, and the primary tumor showed a partial response. Re-growth of primary tumor was, however, reported 8 months after the end of therapy. The patient subsequently received 13 cycles of docetaxel as second-line treatment and 2 cycles of an investigational drug (anti-HER2 [human epidermal growth factor receptor type2] antibody) as third-line treatment. In May 2011, she agreed to participate in the phase 1 study of Compound 1 monotherapy, and she received cabozantinib at a starting dose of 40 mg once a day. Yamamoto N, Nokihara H, Wakui H, Yamada Y, Frye J, DeCillis A, Tamura T. A phase 1 multiple ascending dose study of cabozantinib (XL184) monotherapy in Japanese patients with advanced solid tumors. Molecular Cancer Therapeutics. 2011 10 suppl 1 (abstr C26).

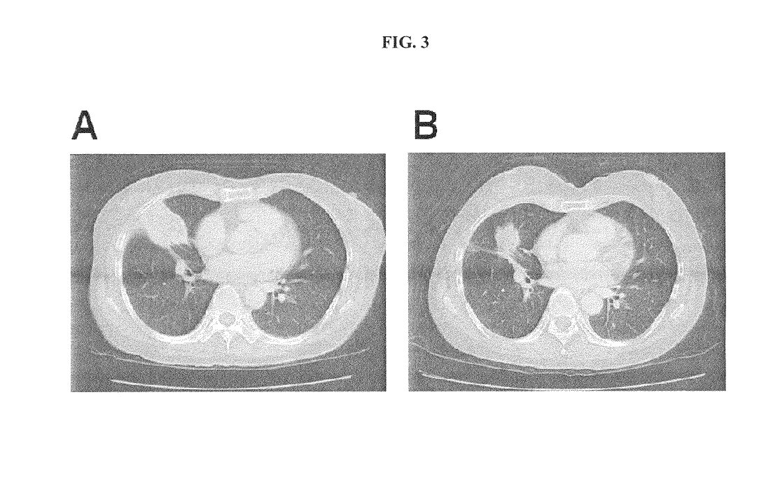

Chest CT scans at 9 weeks demonstrated partial response (40.1% tumor reduction) of her primary lung tumor (FIG. 3), which was subsequently confirmed at 17 weeks. During the cycles (months) of cabozantinib therapy, drug interruptions were employed due to grade 3 serum lipase elevations without clinical symptoms of pancreatitis or abnormal findings on abdominal ultrasonography. In February 2012, she terminated cabozantinib monotherapy due to progressive disease.

### Detection of KIF5B-RET Fusion

The presence of KIF5B-RET fusion in this patient was evaluated retrospectively using pre- and post-treatment samples. Genomic DNA was extracted from pleural effusion cells at diagnosis as a pre-treatment sample, and genomic DNA and total RNA were extracted from pleural effusion cells at progression as a post-treatment sample. Genomic DNA was isolated using a QIAamp DNA Mini kit (Qiagen, Valencia, Calif., USA). TRIzol (Invitrogen, Carlsbad, Calif., USA) was used for the extraction of total RNA according to the manufacturer's instructions and quality was examined using a model 2100 bioanalyzer (Agilent Technologies, Santa Clara, Calif., USA). The sample showed RNA Integrity Numbers >6.0.

Total RNA (500 ng) was reverse-transcribed to cDNA using Superscript III Reverse Transcriptase (Invitrogen). cDNA (corresponding to 10 ng total RNA) or 10 ng genomic DNA was subjected to polymerase chain reaction (PCR) amplification using KAPA Taq DNA Polymerase (KAPA Biosystems, Woburn, Mass., USA). The reactions were carried out in a thermal cycler under the following conditions: 40 cycles at 95° C. for 15 sec, 60° C. for 15 sec and 72° C. for 1 min (for reverse transcriptase (RT)-PCR) or 3 min (for genomic PCR), with a final extension for 10 min at 72° C. The gene encoding glyceraldehyde-3-phosphate dehydrogenase (GAPDH) was amplified to estimate the efficiency of cDNA synthesis. The PCR products were directly sequenced in both directions using the BigDye Terminator kit and an ABI 3130×1 DNA Sequencer (Applied Biosystems, Foster City, Calif., USA). This study was approved by the institutional review boards of the National Cancer Center in Tokyo, Japan. The PCR primers used in the present study are shown in Table 2.

TABLE 2
PCR Primers

| | | | | |
|---|---|---|---|---|
| No. | Name | Location | Sequence | Use |

Genomic PCR

| | | | | |
|---|---|---|---|---|
| 1 | KIF5B- | KIF5B | 5′-GGCATTTGACTTGGTGGTAGAT-3′ | PCR |
| | int15-F2.2 | intron 15 | (SEQ ID NO: 1) | |
| 2 | KIF5B- | RET | 5′-TCCAAATTCGCCTTCTCCTA-3′ | PCR |
| | RET-R1 | exon 12 | (SEQ ID NO: 2) | |
| 3 | AD12- | RET | 5′-CCTGGGAACCCACAGTCAAG-3′ | Sequencing |
| | 001Tseq-R1 | intron 11 | (SEQ ID NO: 3) | |

RT-PCR

| | | | | |
|---|---|---|---|---|
| 3 | KIF5B- | KIF5B | 5′-ATTAGGTGGCAACTGTAGAACC-3′ | PCR |
| | 867F | exon 10 | (SEQ ID NO 4) | |
| 4 | RET-2381R | KIF5B | 5′-AGCCACAGATCAGGAAAAGA-3′ | PCR |
| | | exon 12 | (SEQ ID NO: 5) | |
| 5 | KIF5B- | KIF5B | 5′-AGGAAATGACCAACCACCAG-3′ | Sequencing |
| | RET-F1 | exon 15 | (SEQ ID NO: 6) | |
| 6 | GAPDH-F | GAPDH | 5′-CCAAGGTCATCCATGACAAC-3′ | PCR |
| | | exon 7 | (SEQ ID NO: 7) | |
| 7 | GAPDH-R | GAPDH | 5′-CACCCTGTTGCTGTAGCCA-3′ | PCR |
| | | exon 9 | (SEQ ID NO: 8) | |

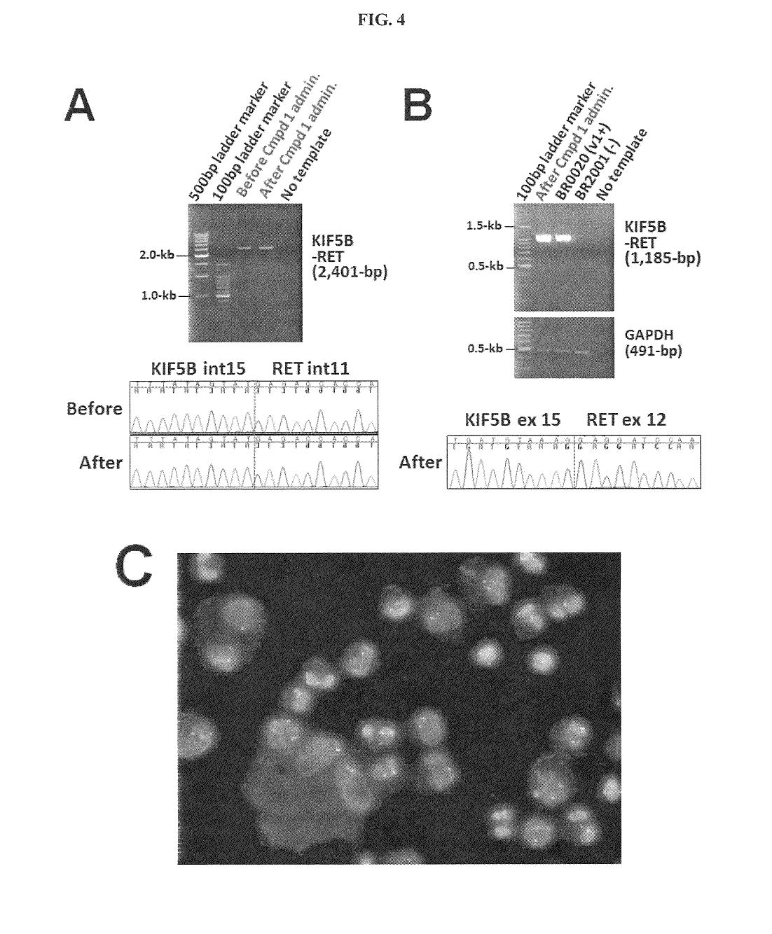

A fusion of the KIF5B (intron 15) and RET (intron 11) genes was detected in genomic DNAs in both pre- and post-treatment samples as depicted in FIG. 4A, which shows KIF5B-RET genome PCR and Sanger sequencing from pre- and post-treatment tumor samples. Sanger sequencing of RT-PCR products verified the expression of variant 1 transcripts (KIF5B exon 15; RET exon 12), the most common type of KIF5B-RET fusion transcripts, in tumor cells, as depicted in FIG. 4B, which shows KIF5B-RET RT-PCR and Sanger sequencing from post-treatment tumor sample. BR0020 (KIF5B-RET variant 1 fusion positive) and BR2001 (KIF5B-RET fusion negative) were used as positive and negative controls. GAPDH (glyceraldehyde-3-phosphate dehydrogenase) transcripts were amplified to estimate the quantity and quality of cDNAs. T, Ichikawa H, Totoki Y, Yasuda K, Hiramoto M, Nammo T, Sakamoto H, Tsuta K, Furuta K, Shimada Y, Iwakawa R, Ogiwara H, Oike T, Enari M, Schetter A J, Okayama H, Haugen A, Skaug V, Chiku S, Yamanaka I, Arai Y, Watanabe S, Sekine I, Ogawa S, Harris C C, Tsuda H, Yoshida T, Yokota J, Shibata T. KIF5B-RET fusions in lung adenocarcinoma. Nat. Med. 2012 Feb. 12; 18(3):375-7. Takeuchi K, Soda M, Togashi Y, Suzuki R, Sakata S, Hatano S, Asaka R, Hamanaka W, Ninomiya H, Uehara H, Lim Choi Y, Satoh Y, Okumura S, Nakagawa K, Mano H, Ishikawa Y. RET, ROS1 and ALK fusions in lung cancer. Nat. Med. 2012 Feb. 12; 18(3):378-81. Lipson D, Capelletti M, Yelensky R, Otto G, Parker A, Jarosz M, Curran J A, Balasubramanian S, Bloom T, Brennan K W, Donahue A, Downing S R, Frampton G M, Garcia L, Juhn F, Mitchell K C, White E, White J, Zwirko Z, Peretz T, Nechushtan H, Soussan-Gutman L, Kim J, Sasaki H, Kim H R, Park S I, Ercan D, Sheehan C E, Ross J S, Cronin M T, Janne P A, Stephens P J. Identification of new ALK and RET gene fusions from colorectal and lung cancer biopsies. Nat. Med. 2012 Feb. 12; 18(3):382-4. Cytological materials derived from the pre-treatment pleural effusion sample underwent fluorescent in situ hybridization (FISH) analysis using a break-apart RET probe set (Chromosome Science Labo Inc, Sapporo, Japan), which hybridizes with the neighboring 5′ centromeric (RP11-379D20, labeled with Spectrum Green) and 3′ telomeric (RP11-875A4, labeled with Spectrum Red) sequence of the RET gene as depicted in FIG. 4C, which shows break-apart FISH at the RET locus. Tumor cells show split (5′ green and 3′ orange) signals in addition to fused signals (original magnification, 100×). A split signal defined by 5′ and 3′ probes observed at a distance>1 times the signal size was observed in tumor cells. Thus, the tumor was judged to have a rearrangement of the RET gene, consistent with the PCR results above.

### Discussion

This is the first reported case in which a RET TKI has shown marked antitumor activity in a patient with KIF5B-RET fusion-positive NSCLC. To date, in vitro studies revealed that the growth and signaling properties mediated by KIF5B-RET were diminished after treatment with TKIs such as vandetanib, sunitinib or sorafenib. However, there has been no report that a patient with KIF5B-RET fusion-positive NSCLC responded to these drugs. Our report suggests that patients with advanced NSCLC harboring KIF5B-RET fusion may be exquisitely sensitive to therapeutic RET inhibition.

We have identified that approximately 2% of NSCLC patients harbor KIF5B-RET fusion. KIF5B-RET fusion-positive NSCLC comprises only a small subset of all lung cancers, however, lung cancer is a common disease and the number of lung cancer patients is increasing annually, so this subset translates into a considerable number of patients world-wide Therefore, the authors recommend development of a systematic screening method to identify KIF5B-RET fusion-positive NSCLC. The discovery of EML4-ALK rearrangements in NSCLC was published in 2007 and the US Food and Drug Administration approved crizotinib for this disease in 2011, followed by approval in Japan in 2012. Soda M, Choi Y L, Enomoto M, Takada S, Yamashita Y, Ishikawa S, Fujiwara S, Watanabe H, Kurashina K, Hatanaka H, Bando M, Ohno S, Ishikawa Y, Aburatani H, Niki T, Sohara Y, Sugiyama Y, Mano H. Identification of the transforming EML4-ALK fusion gene in non-small-cell lung cancer. Nature. 2007 Aug. 2; 448(7153):561-6. This KIF5B-RET-positive patient had a marked clinical response to Compound 1, and this finding suggests that KIF5B-RET fusion is a driver oncogene in NSCLC and a promising therapeutic target.

Compound 1 is a potent inhibitor of TK against RET, a kinase that has been implicated in tumor pathobiology. For example, Yakes discloses that Compound 1 exhibits strong inhibition of RET, with an IC₅₀ of 5.2±4.3 nMol/L. Yakes F M, Chen J, Tan J, Yamaguchi K, Shi Y, Yu P, Qian F, Chu F, Bentzien F, Cancilla B, Orf J, You A, Laird A D, Engst S, Lee L, Lesch J, Chou Y C, Joly A H. Cabozantinib (XL184), a novel MET and VEGFR2 inhibitor, simultaneously suppresses metastasis, angiogenesis, and tumor growth. Mol Cancer Ther. 2011 December; 10(12):2298-308. Activating mutations in RET play an important role in tumorigenesis in medullary thyroid cancer (MTC). Sennino B, Naylor R M, Tabruyn S P, You W K, Aftab D T McDonald D M. Reduction of tumor invasiveness and metastasis and prolongation of survival of RIP-Tag2 mice after inhibition of VEGFR plus c-Met by XL184. Mol Cancer Ther. 2009 8 suppl 1 (abstr A13). In a phase I dose-escalation study of cabozantinib, 25 (68%) of 37 patients with MTC had a confirmed partial response or stable disease for 6 months or longer. Kurzrock R, Sherman S I, Ball D W, Forastiere A A, Cohen R B, Mehra R, Pfister D G, Cohen E E, Janisch L, Nauling F, Hong D S, Ng C S, Ye L, Gagel R F, Frye J, Müller T, Ratain M J, Salgia R. Activity of XL184 (Cabozantinib), an oral tyrosine kinase inhibitor, in patients with medullary thyroid cancer. J Clin Oncol. 2011 Jul. 1; 29(19):2660-6. In this study tumor regression was observed in patients with and without known RET mutations, suggesting some responses were caused by inhibition of targets other than RET, such as MET and/or VEGFR2, or as yet unknown aberrations in the RET pathway.

In summary, our NSCLC patient with KIF5B-RET fusion had a clinical response to cabozantinib, indicating that cabozantinib may be active in patients with advanced NSCLC harboring KIF5B-RET fusion. Urgent clinical evaluation of RET-TKIs against KIF5B-RET fusion-positive NSCLC is warranted.

### Other Embodiments

The foregoing disclosure has been described in some detail by way of illustration and example, for purposes of clarity and understanding. The invention has been described with reference to various specific and preferred embodiments and techniques. However, it should be understood that many variations and modifications can be made while remaining within the spirit and scope of the invention. It will be obvious to one of skill in the art that changes and modifications can be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

1. A method for treating lung adenocarcinoma, comprising administering to a patient in need of such treatment an effective amount of a compound of Formula I: 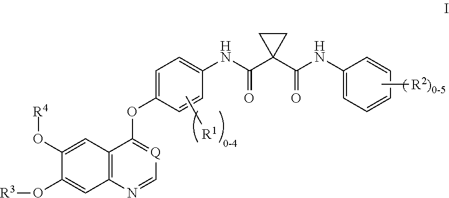
or a pharmaceutically acceptable salt thereof, wherein:
R¹ is halo;
R² is halo;
R³ is (C₁-C₆)alkyl;
R⁴ is (C₁-C₆)alkyl; and
Q is CH or N.

2. The method of claim 1, wherein the lung adenocarcinoma is non-small cell lung cancer.

3. The method of claim 1, wherein the lung adenocarcinoma is KIF5B-RET fusion-positive non-small cell lung cancer.

4. The method of claim 2, wherein the compound of Formula I is a compound of Formula Ia 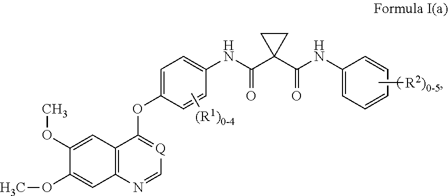
or a pharmaceutically acceptable salt thereof, wherein:
R¹ is halo;
R² is halo; and
Q is CH or N.

5. The method of claim 4, wherein the compound of Formula I is compound 1 which is N-(4-{[6,7-bis(methyloxy)quinolin-4-yl]oxy}phenyl)-N′-(4-fluorophenyl)cyclopropane-1,1-dicarboxamide: 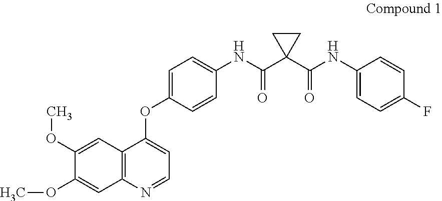 [SMILES: COc1cc2nccc(Oc3ccc(NC(=O)C4(C(=O)Nc5ccc(F)cc5)CC4)cc3)c2cc1OC]
or a pharmaceutically acceptable salt thereof.

6. The method of claim 5, wherein compound 1 is N-(4-{[6,7-bis(methyloxy)quinolin-4-yl]oxy}phenyl)-N′-(4-fluorophenyl)cyclopropane-1,1-dicarboxamide.

7. The method of claim 6, wherein compound 1 is the (L)- or (D)-malate salt.

8. The method of claim 7, wherein the compound of Formula (I) is in the crystalline N-1 or N-2 form of the (L) malate salt and/or the (D) malate salt.

9. The method of claim 8 wherein compound 1, or a pharmaceutically acceptable salt thereof, is administered as a pharmaceutical composition additionally comprising a pharmaceutically acceptable carrier, excipient, or diluent.

10. The method of claim 9, wherein compound 1 is administered subsequent to another form of treatment.

11. The method of claim 9, wherein compound 1 is administered post-cisplatin and/or gemcitabine treatment.

12. The method of claim 9, wherein compound 1 is administered post-doectaxel treatment.

13. The method of claim 9, wherein the compound of Formula I is administered post-cisplatin and/or gemcitabine and/or docetaxel treatment.

14. A method for treating lung adenocarcinoma which is KIF5B-RET fusion-positive non-small cell lung cancer in a patient in need of such treatment comprising administering a therapeutically effective amount of compound 1 or a pharmaceutically acceptable salt thereof.

15. A method for inhibiting or reversing the progress of abnormal cell growth in a mammal, comprising administering to the mammal an effective amount of compound 1 or a pharmaceutically acceptable salt thereof, wherein the abnormal cell growth is cancer mediated by KIF5B-RET.

16. The method of claim 15, wherein the cancer is lung adenocarcinoma.

17. The method of claim 15, wherein the lung adenocarcinoma is non-small cell lung cancer.

18. The method of claim 15, wherein the lung adenocarcinoma is KIF5B-RET fusion-positive non-small cell lung cancer.

19. The method of claim 18, wherein Compound 1 or a pharmaceutically acceptable salt thereof is administered as a pharmaceutical composition comprising Compound 1 or a pharmaceutically acceptable salt thereof and at least one pharmaceutically acceptable carrier.

20. A method for treating a lung adenocarcinoma which is KIF5B-RET fusion positive non-small cell lung cancer in a patient in need of such treatment, comprising administering to the patient an effective amount of compound 1: 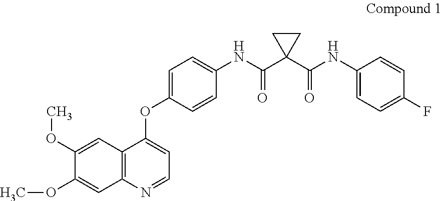 [SMILES: COc1cc2nccc(Oc3ccc(NC(=O)C4(C(=O)Nc5ccc(F)cc5)CC4)cc3)c2cc1OC]
or a pharmaceutically acceptable salt thereof.


SEQUENCE LISTING (8 sequences)

SEQ ID NO: 1 - DNA, 22 bp, Artificial Sequence
  Synthetically generated sequence
ggcatttgacttggtggtagat

SEQ ID NO: 2 - DNA, 20 bp, Artificial Sequence
  Synthetically generated sequence
tccaaattcgccttctccta

SEQ ID NO: 3 - DNA, 20 bp, Artificial sequence
  Synthetically generated sequence
cctgggaacccacagtcaag

SEQ ID NO: 4 - DNA, 22 bp, Artificial Sequence
  Synthetically generated sequence
attaggtggcaactgtagaacc

SEQ ID NO: 5 - DNA, 20 bp, Artificial Sequence
  Synthetically generated sequence
agccacagatcaggaaaaga

SEQ ID NO: 6 - DNA, 20 bp, Artificial Sequence
  Synthetically generated sequence
aggaaatgaccaaccaccag

SEQ ID NO: 7 - DNA, 20 bp, Artificial Sequence
  Synthetically generated sequence
ccaaggtcatccatgacaac

SEQ ID NO: 8 - DNA, 19 bp, Artificial Sequence
  Synthetically generated sequence
caccctgttgctgtagcca